(19) United States
(12) Patent Application Publication
(10) Pub. No.: US 2026/0140102 A1
(43) Pub. Date: May 21, 2026

(54) IMPEDANCE MEASUREMENT USING BIPOLAR CONFIGURATIONS OF ELECTRODES

(71) Applicant: NOVASCAN, INC., Chicago, IL (US)

(72) Inventor: Donato M. CERES, Chicago, IL (US)

(21) Appl. No.: 18/949,077

(22) Filed: Nov. 15, 2024

(51) Int. Cl. G01N 33/483; G01N 27/02

(52) U.S. Cl. CPC G01N 33/4836; G01N 27/02

(57) ABSTRACT

Various embodiments disclose system for determining electrical properties of tissue samples, the system comprising a tissue measurement tool comprising at least two electrodes that measure, while operating at a frequency, a set of electrical properties corresponding to a section of tissue, and a decoupling device connected to the at least two electrodes, and a classification module that computes, based on the set of electrical properties, at least an electrical impedance of the section of tissue.

### BACKGROUND

### Field of the Various Embodiments

The various embodiments relate generally to electronics and medical diagnostic technology and, more specifically, to impedance measurement at high frequencies using electrodes.

### Description of the Related Art

In various medical procedures, tissue cells are tested and analyzed to make various diagnoses for a patient. Histological assessment of frozen sections of tissue samples is a common intraoperative technique for rapidly assessing margins during resection of cancer in tissues of the breast, prostate, lung, ovarian, cervical, and skin. For example, Mohs micrographic surgery (MMS) is a skin-sparing cancer removal surgery often used when the lesion is particularly visible, like on the face. During MMS, a surgeon removes a layer of skin from a target area of the patient, where the skin within the target area is suspected of including cancer cells. Immediately after removing the excised layer, frozen sections are prepared, and the surgeon examines slides under a microscope to determine whether cancer cells are, in fact, present within a given margin of the inner part of the tissue. According to the overarching procedure, the surgeon successively removes and examines skin layers from the patient until no cancer cells are present within a satisfactory margin of the frozen sections. The frozen sections of tissue advantageously enable a surgeon to properly analyze the margin of an excised skin sample and subsequently determine if residual cancer is still present in the patient.

Other rapid intraoperative assessments of excised tissue are also critical in endoscopically removed biopsies that use forceps or fine needles. Due to the difficulty of locating a lesion accurately using an endoscope, fast feedback and assessment of a biopsy sample are thus required to help the surgeon understand if the sample is representative of the lesion of concern. Typical intraoperative assessment of biopsy samples is done by ROSE (Rapid On-Site Evaluation). In ROSE, a small number of cells are released on microscope slides by smearing biopsy samples (touch-prep), followed by staining to similar frozen sections for histological assessment. Biopsy samples continue to be collected until a satisfactory diagnosis has been reached. ROSE advantageously enables surgeons to properly analyze the biopsy samples, increase the accuracy of the diagnosis, and help find a suitable treatment for the patient.

Margin assessments using frozen sections and evaluation of the samples by ROSE are relatively fast and convenient. However, the overall process for margin assessment of frozen cells remains relatively slow due to the necessity to prepare the samples. For example, the frozen sections must be properly mounted and sliced before going through the staining process. As a result, the entire preparation procedure is typically performed over a period of 20 to 40 minutes. Further, touch-prep and staining take several minutes. Moreover, the ability to release enough cells, if any at all, on the microscope slide can prevent the proper assessment of the sample, resulting in increased diagnostic uncertainty of the lesion.

More recently, various tissue measurement systems have been incorporated into medical procedures to perform faster diagnostic measurements of tissue margins using electrical techniques. For example, various tissue measurement systems have been developed to perform bioelectrical impedance analysis (BIA) and bioelectrical impedance spectroscopy (BIS) on excised tissue. Surgeons have incorporated tissue measurement systems intraoperatively to determine the electrical properties of excised tissue and detect the presence of cancerous cells within the excised tissue in lieu of freezing, transferring cells to a microscope slide, and manually examining the excised tissue under a microscope. Electrical techniques characteristically can access sufficient amounts of tissue to analyze, as the measurement systems typically can analyze very small quantities of tissue. Further, the measurement systems can analyze the tissue as-is such that the tissue does not need to be destroyed and can be processed in additional ways. Further, electrical techniques executed by the measurements systems can take only a few seconds to assess a sample instead of minutes.

At least one drawback of conventional measurement systems is that the wiring required to connect the electrodes in the can introduce substantial parasitic impedances that can adversely impact the quality of the voltage and current measurements that are acquired. For example, parasitic impedances frequently degrade the accuracy of the voltages and currents that are measured by conventional electrical tissue measurement systems at high frequencies, which limits the frequency range in which conventional electrical measurement systems can operate effectively. Notably, though, various types of cancerous cells exhibit electrical impedances that are distinct from noncancerous cells only at high frequencies. Because many, if not most, conventional electrical measurement systems cannot accurately measure impedances at higher frequencies due to intrinsic parasitic impedances, these conventional measurement systems are not able to distinguish certain types of cancerous cells from noncancerous cells, thereby reducing the overall usefulness and effectiveness of conventional measurement systems.

Another drawback of conventional measurement systems is that many systems require large and complex circuitry to acquire the voltage and current measurements necessary to compute the electrical impedance of cells within excised tissue accurately. For example, many conventional electrical measurement systems require multiple electrodes in certain configurations (e.g., tetrapolar configurations including four electrodes) to measure the voltage and current associated with a given section of the excised tissue. As a result, the tissue still needs to span over the multiple electrodes included in the configuration to make proper contact and for subsequent analysis. Further, conventional electrical measurement systems impose a minimum size requirement due to the restrictions imposed by the configuration of the electrodes, limiting how the electrical measurement system can be miniaturized and thus limiting how the electrical measurement system can be included in in-vivo devices.

As the foregoing illustrates, what is needed in the art are more effective techniques to analyze electrical impedances of tissue.

### SUMMARY

Various embodiments disclose system for determining electrical properties of tissue samples, the system comprising a tissue measurement tool comprising at least two electrodes that measure, while operating at a frequency, a set of electrical properties corresponding to a section of tissue, and a decoupling device connected to the at least two electrodes, and a classification module that computes, based on the set of electrical properties, at least an electrical impedance of the section of tissue.

At least one technical advantage of the disclosed design relative to the prior art is that the disclosed design enables a tissue measurement system to be employed for bioelectrical impedance spectroscopy over a wider range of frequencies relative to the effective frequency ranges of conventional tissue measurement systems. In particular, the disclosed design includes a decoupling device that enables the removal of parasitic impedances, which are otherwise generated by the components of the device and electrodes, from the voltages and currents measured by a tissue measurement tool. A tissue measurement system that incorporates the disclosed design can achieve greater impedance accuracy over a wider range of frequencies, such as frequencies above 2 MHz to 100 MHz. Consequently, a tissue measurement system that incorporates the disclosed design can thus distinguish between additional types of cancerous cells and noncancerous cells. Further, various embodiments of the disclosed design use bipolar configurations of electrodes in an electrode array. A tissue measurement system that incorporates the disclosed design thus requires fewer electrodes when measuring the voltages and currents associated with given tissue samples, enabling the electrode array to be miniaturized and used in a wider range of environments. These technical advantages provide one or more technological improvements over prior art approaches.

### BRIEF DESCRIPTION OF THE DRAWINGS

So that the manner in which the above recited features of the various embodiments can be understood in detail, a more particular description of the inventive concepts, briefly summarized above, may be had by reference to various embodiments, some of which are illustrated in the appended drawings. It is to be noted, however, that the appended drawings illustrate only typical embodiments of the inventive concepts and are therefore not to be considered limiting of scope in any way, and that there are other equally effective embodiments.

FIG. 1 illustrates a tissue measurement system configured to implement one or more aspects of the present disclosure;

FIG. 2 illustrates how the electrodes of the tissue measurement system of FIG. 1 measure tissue impedance, according to various embodiments;

FIG. 3 illustrates a bipolar configuration for a pair of electrodes that can be included in the tissue measurement system of FIG. 1, according to various embodiments;

FIG. 4 illustrates exemplar configurations for a decoupling device that can be coupled to the electrodes of the tissue measurement system of FIG. 1, according to various embodiments;

FIG. 5 is a graph illustrating different features extracted from the tissue impedances measured by a tissue management tool that does not include a decoupling device, according to various embodiments;

FIG. 6 is a graph illustrating different features extracted from the tissue impedances measured by a tissue management tool that includes a decoupling device, according to various embodiments, according to various embodiments;

FIG. 7 sets forth a flowchart of method steps for measuring tissue impedances using a bipolar electrode configuration, according to various embodiments; and

FIG. 8 sets forth a flowchart of method steps for measuring tissue impedances using a bipolar electrode configuration and a decoupling device having variable impedance, according to various embodiments.

### DETAILED DESCRIPTION

In the following description, numerous specific details are set forth to provide a more thorough understanding of the various embodiments. However, it will be apparent to one skilled in the art that the inventive concepts may be practiced without one or more of these specific details. For explanatory purposes, multiple instances of like objects are symbolized with reference numbers identifying the object and parenthetical numbers(s) identifying the instance where needed.

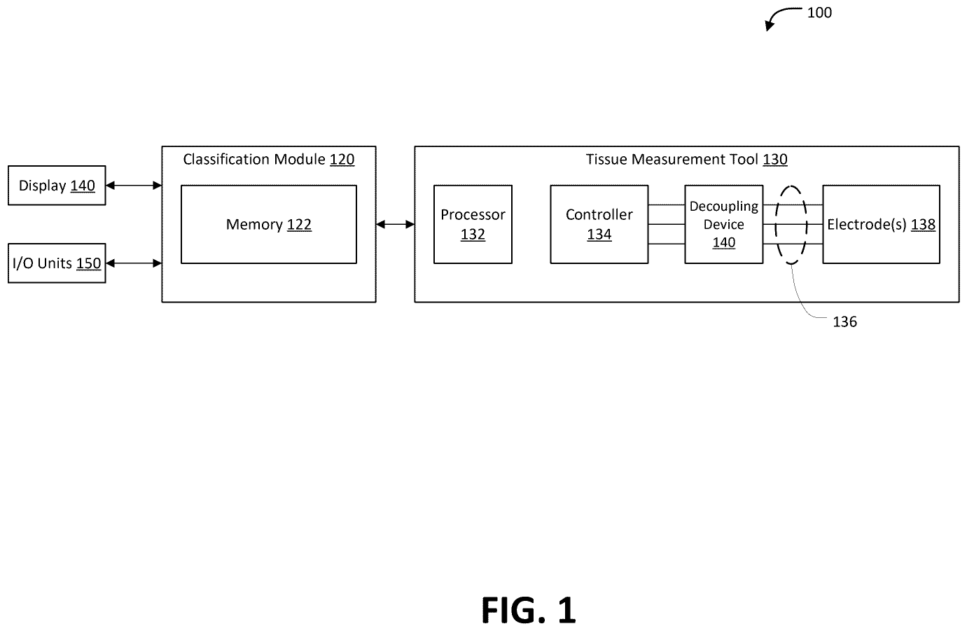

FIG. 1 illustrates a tissue measurement system 100 configured to implement one or more aspects of the present disclosure. As shown, the tissue measurement system 100 includes, without limitation, a classification module 120, a tissue measurement tool 130, a display 140, and one or more input/output (I/O) units 150. The classification module 120 includes, without limitation, a memory 122. The tissue measurement tool 130 includes, without limitation, a processor, a controller 134, one or more connectors 136, one or more electrodes 138, and a decoupling device.

The tissue measurement tool 130 automatically measures a voltage and a current across a section of a tissue sample. In some embodiments, the controller 134 causes the electrodes 138 to measure the electrical properties of a different section of the tissue sample. In some embodiments, when performing measurements on the section of the tissue sample, the controller 134 can perform a sweep of measurements within a range of operating frequencies. For example, an electrode 138 (e.g., a first electrode 138(1)) can initially inject a current at an initial operating frequency between 1 kHz and 25 MHz. The tissue measurement tool 130 can then measure the voltage and current across the section of the tissue sample and determine corresponding electrical properties. The tissue measurement tool 130 can then sweep through a range of operating frequencies. For example, the tissue measurement tool 130 can increase the operating frequency of the injecting current at steps of 1 kHz.

In some embodiments, the tissue measurement tool 130 may include the processor 132, the controller 134, the connectors 136, and/or the electrodes 138 as separate physical components. In alternative embodiments, the processor 132, the controller 134, the connectors 136, the electrodes 138, the classification module 120, the display 140, and/or the I/O units 150 can share a common housing. In some embodiments, the tissue measurement tool 130 and/or the classification module 120 can communicate wirelessly with the display 140 and/or the I/O units 150.

In various embodiments, the tissue measurement tool 130 includes a plurality of electrodes 138. For example, the electrodes 138 can be includes in an electrode array, where the electrode array (e.g., a grouping of 2 or more electrodes 138) includes electrodes 138 that are electrically isolated from each other by intervening channels. In some embodiments, the electrodes 138 in the electrode array are planar, allowing an excised tissue sample to be placed directly on two or more electrodes 138 of the electrode array. In some embodiments, the electrodes 138 are arranged in an interdigitated configuration. Additionally, or alternatively, in some embodiments, the electrodes 138 are arranged as electrode pairs, where the electrode pairs alternate orientation (e.g., a first electrode pair 138(1)-138(2) oriented at 0° and a second electrode pair 138(3)-138(4) oriented at 90°). In some embodiments, one or more of the electrodes 138 included in the electrode array are non-invasive and can have a surface configured to reduce electrical polarization between the individual electrodes 138 and the tissue sample. For example, one or more of the electrodes 138 can have, but is not limited to, a blackened platinum (BPt) or silver/silver chloride (Ag/AgCl) surface that physically contacts a portion of the tissue sample, reducing the electrical polarization between the one or more electrodes 138 and the tissue sample.

In various embodiments, a set of connectors 136 connect electrical signals between the electrodes 138 and the controller 134. As will be discussed in further detail below, in various embodiments, the set of connectors 136 can be combined to connect to an electrode pair (e.g., electrodes 138(1)-138(2)) from multiple electrodes 138, where the electrode pair is connected in a bipolar configuration. In such instances, the controller 134 can control and/or switch which electrodes 138 are connected to the set of connectors 136.

The controller 134 connects through the set of connectors 136 to the electrodes 138. In some embodiments, the controller 134 responds to commands from the classification module 120 to conduct multiple electrical measurements on the tissue sample using the electrodes 138. In some embodiments, the controller 134 responds to the commands by selecting different electrodes 138 in an electrode array to measure voltages and currents through different sections of the tissue sample. In such instances, the controller 134 can drive a current through different subsets of the set of connectors 136 to inject the current into different electrodes 138. In some embodiments, when the electrode array includes a plurality of electrodes 138, the controller 134 can also include a switching mechanism (not shown) that controls which electrodes 138 are used. For example, the controller 134 via the switching mechanism can select two electrodes 138 at any given time to connect to the connectors 138 to measure electrical properties of the tissue sample. The classification module 120 can then determine the impedance of the tissue sample based on the measured electrical properties. In some embodiments, the switching mechanism includes relays (e.g., motorized relays and/or mechanical relays) and/or solid-state multiplexers to switch the selection of electrodes 138 that are connected to the connectors 136.

In some embodiments, the tissue measurement tool 130 includes a plurality of controllers 134. In such instances, each controller 134 (e.g., 134(1), 134(2), etc.) in the plurality of controllers 134 is connected to at least one pair of electrodes 138. In various embodiments, the plurality of controllers 134 can perform measurements of the tissues in contact with electrodes 138 sequentially or in parallel. The processor 132 can receive the measurements from controllers 134 and process the electrical properties according to the system configuration.

In various embodiments, the controller 134 sets the operating frequency and amplitude of the injection current when initiating a measurement. In some embodiments, the classification module 120 can load instructions stored in memory 122 and execute a measurement program to generate commands for the controller 134. In such instances, the classification module 120 generates and transmits one or more commands to the controller 134 to connect to different measuring subsets of an electrode array. The controller 134 can then change to a different measuring subset by connecting a specific pair of electrodes 138 to the measuring circuit, while disconnecting all remaining electrodes 138 in the electrode array. Additionally, or alternatively, the controller 134 can respond to commands received from the classification module 120 by driving the electrodes 138 to physically move to a new location to measure a different section of the tissue sample.

In some embodiments, the tissue measurement tool 130 can include a processor 132. The processor 132 can be a single central processing unit (CPU), or combination of processing units. The processor 132 can be any technically-feasible hardware unit capable of processing data and/or executing software code. In some embodiments, the processor of the tissue measurement tool 130 can receive instructions from a user or commands from an application stored in the memory 122 of the classification module 120 and can execute those instructions or commands. In some embodiments, the processor 132 can implement one or more techniques executed by the classification module 120.

In various embodiments, the classification module 120 receives instructions from a user via the I/O units 150 to store data or to perform specific electrical measurements via the tissue measurement tool 130. In some embodiments, the classification module 120 can store the electrical properties determined by the tissue measurement tool 130, such as the measured voltage and/or the measured current that are based on an input signal at a specific operating frequency when connected to a tissue sample. In various embodiments, the classification module 120 computes real and imaginary impedances for the testing environment (e.g., a total impedance Zₘ) based on the electrical properties. In some embodiments, the classification module 120 stores the computed impedances in the memory 122. Additionally, or alternatively, in some embodiments, the classification module 120 receives electrical measurements of the tissue measurement tool 130 when disconnected from a tissue sample. In such instances, the classification module 120 computes real and imaginary impedances for the tissue measurement tool 130 (e.g., a tissue measurement tool impedance Z_{d}). The classification module 120 can then compute the real and imaginary impedances for the tissue (e.g., a tissue impedance Zₜ) based on the total impedance Zₘ and the tissue measurement tool impedance Zₜ.

The memory 122 is configured to store data and/or software applications. The memory 122 can include a random-access memory (RAM) module, hard disk, flash memory unit, or any other type of memory unit or combination thereof. In various embodiments, the classification module 120, the tissue measurement tool 130, and/or the I/O units 150 are configured to read data from the memory 122. The classification module 120, the tissue measurement tool 130, and/or the I/O units 150 are also configured to write data to memory 122. In some embodiments, the memory 122 stores a tissue analysis application that includes instructions that are executed by the processor 132 to control the tissue measurement tool 130 measuring electrical properties and/or the classification module 120 computing corresponding values, such as various electrical impedances.

The display 140 displays data transmitted from the classification module 120. In various embodiments, the display 140 displays one or more electrical measurements and/or computed electrical properties (e.g., tissue impedances, etc.). In some embodiments, the display 140 shows the estimated location(s) of cancerous cell regions and the probability of cancer in the tissue sample. In some embodiments, the display 140 may refresh the data received from the classification module 120 while the tissue measurement tool 130 performs measurements on the tissue sample. In some embodiments, the display 140 may display an image of the tissue sample with indications of the locations of probable cancerous cells. In some embodiments, when the electrodes 138 do not acquire a map of the tissue sample or do not discover the location of a target (e.g., the location of cancer within the tissue sample), the display 140 can display the computed single impedance of the tissue and/or the probability of the tissue sample containing cancerous cells.

The I/O units 150 receive output signals from the tissue measurement tool 130 and/or the classification module 120 and transmit input signals from a user to the tissue measurement tool 130 and/or the classification module 120. In some embodiments, the I/O units 150 transmit program input signals the classification module 120, where the classification module 120 stores the program in the memory 122. In some embodiments, the I/O units 150 can include devices capable of receiving one or more inputs, including a keyboard, mouse, input tablet, camera, and/or three-dimensional (3D) scanner. In some embodiments, the I/O units 150 can also include devices capable of providing one or more outputs, such as a speaker or printer. The I/O units 150 can also include devices capable of both receiving inputs and providing outputs, such as a touchscreen and a universal serial bus (USB) port.

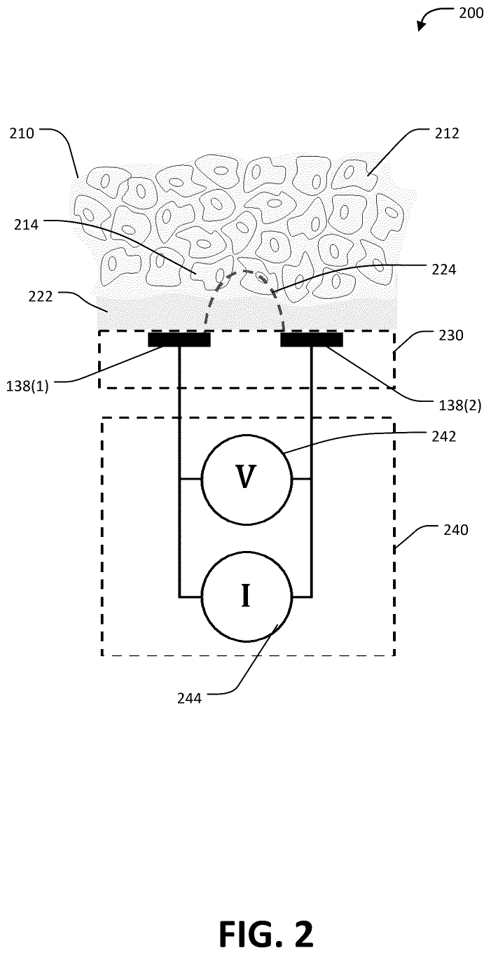

FIG. 2 illustrates how the electrodes of the tissue measurement system 100 of FIG. 1 measure tissue impedance, according to various embodiments. As shown, the testing environment 200 includes, without limitation, a tissue sample 210, an interface 222, an electrode array 230, and a measuring circuit 240. The tissue sample 210 includes, without limitation, one or more healthy cells 212 and one or more cancerous cells 214. The electrode array 230 includes, without limitation, an electrode pair including the electrodes 138(1)-138(2). The measuring circuit 240 includes, without limitation, a voltage-sensing device 242 and a current generator 244.

During operation, the controller 134 measures the electrical properties of a section of the tissue sample 210 by selecting the electrode pair formed by the electrodes 138(1)-138(2) from the electrode array 230. The measuring circuit 240 transmits a current through the electrode array 230 and the interface 222 to the tissue sample 210. The current generates a current field 224 that flows through a section of the tissue sample 210. The measuring circuit 240 measures the voltage and current passing from the electrode 138(1) to the electrode 138(2) through the tissue sample 210. Based on the voltage and current measurements, the tissue measuring system 100 can determine the tissue impedance Zₜ for the tissue sample 210 and whether the tissue sample includes any cancerous cells 214 amongst the healthy cells 212.

In some embodiments, the controller 134 can measure varying depths of the tissue sample 210 by selecting an electrode pair formed from electrodes 138 that are located further away from the section (e.g., different portions of an interdigitated electrode array). For example, when the controller 134 connects the pair of electrodes 138(1)-138(2) to the measuring circuit 240, the electrodes 138(1)-138(2) are connected in a bipolar configuration to both a voltage-sensing device and to a current-sensing device, while the remainder of electrodes in the electrode array 230 are disconnected from the measuring circuit 240.

In various embodiments, the controller 134 can measure the tissue sample 210 in bulk, including instances where the size of the tissue sample 210 is greater than the distance between two adjacent electrodes 138. In such instances, the controller 134 can select one or more electrode pairs formed from electrodes 138 that are configured in an interdigitated electrode array. In the interdigitated electrode array, anodes and cathodes are alternated, all anodes are connected together, and all cathodes are connected together. The interdigitated electrode array is connected to the measuring circuit 240 in a bipolar configuration. In such instances, the controller 134 does not select specific electrode pairs of varying distances (e.g., selecting electrodes located further away to measure a specific depth within the tissue sample 210). The resulting measurements reflect a global measurement of the entire tissue sample 210 that is in contact with the interdigitated electrode array.

In various embodiments, the electrode pair 138(1)-138(2) are included in a current-sensing circuit. In such instances, the electrode 138(1) acts as an injection electrode (e.g., a source electrode) that receives a current from a current generator 244. The injection electrode 138(1) receives an alternating current that has a frequency corresponding to the operating frequency specified by the controller 134. The electrode 138(2) acts as a return electrode (e.g., a drain electrode) that completes a current path via the current field 224 by connecting to electrode 138(1). In some embodiments, the return electrode 138(2) is connected to a current-sensing circuit or current-sensing device, such as an ammeter, included in the measuring circuit 240. In various embodiments, the classification module 120 receives the current measurement provided by the current-sensing circuit or current-sensing device and associates the measured current with the operating frequency of the initial current generated by the current generator 244.

In some embodiments, the electrodes 138(1)-138(2) of the electrode pair are part of a voltage-sensing device 242 that includes a voltage source. For example, the electrodes 138(1)-138(2) can be connected to the measuring circuit 240 in a bipolar configuration. In such instances, the electrodes 138(1)-138(2) can also act as voltage-sensing electrodes and be connected to a voltage-sensing circuit or a voltage-sensing device, such as a voltmeter, included in the measuring circuit 240. In some embodiments, the electrodes 138(1)-138(2) can have high impedances in order to avoid adding stray currents into the measuring circuit. In various embodiments, the classification module 120 can receive the measured voltage provided by the voltage-sensing circuit or voltage-sensing device and associate the measured current with the operating frequency of the initial current.

In some embodiments, the tissue measurement tool 130 can measure different sections of the tissue sample 210 by switching to different electrode pairs (not shown). For example, the controller 134 may cause the measuring circuit 240 to disconnect from the electrodes 138(1)-138(2) and connect to a different electrode pair in the electrode array 230. In some embodiments, the controller 134 may switch between electrodes 138 in the electrode array 230 in a pre-defined pattern. For example, the controller 134 may perform a series of electrical measurements on a section of the tissue sample 210 for 10 to 60 seconds. The controller 134 may then cause the measuring circuit 240 to connect to a different electrode pair to perform an additional series of electrical measurements on a different section of the tissue sample 210 for 10 to 60 seconds. In some embodiments, the controller 134 can measure electrical properties for all sections of the tissue sample 210 within the electrode array 230 in under 60 to 120 seconds. In some embodiments, each pair of electrodes 138 included in the electrode array 230 is connected to a separate measuring circuit 240 (e.g., measuring circuits 240(1), 240(2), etc.). In such instances the processor 132 obtains and processes the impedances of different portions of the tissue sample 210.

In various embodiments, the classification module 120 computes electrical impedances based on the measured electrical properties of sections of the tissue sample 210 and subsequently computes a Cole relaxation frequency (F_{cole}) from the computed electrical impedances. The Cole relaxation frequency for a section of the tissue sample 210 reflects the rate at which a cell membrane of a healthy cell 212 or a cancerous cell 214 discharges a stored electrical charge. In some embodiments, the tissue measurement tool 130 computes the Cole relaxation frequency as an average of electrical discharge rates for a plurality of cells 212, 214 included in the section of the tissue sample 210.

In some embodiments, the tissue measurement system 100 can determine the presence and/or location of the cancerous cells 214 within the section of the tissue sample 210 based on computing Cole relaxation frequencies for one or more sections of the tissue sample 210. Due to the contrasting electrical properties of certain cancerous cells 214 and healthy cells 212, the cancerous cells 214 have a Cole relaxation frequency that is over one thousand times smaller than the Cole relaxation frequency of the healthy cell 212. In such instances, the classification module 120 compares the computed Cole relaxation frequency to a pre-determined cancer-detection threshold to determine whether the section of the tissue sample 210 contains cancerous cells 214. Additionally, or alternatively, the contrasting electrical properties of cancerous cells 214 and healthy cells 212 also means that cancerous cells 214 have higher electrical impedances than healthy cells 212 when the current is at a high frequency (e.g., above 10-100 MHz). In such instances, the classification module 120 compares the electrical impedances to a pre-determined cancer-detection threshold for the frequency range to determine whether the section of the tissue sample 210 contains cancerous cells 214.

In various embodiments, the classification module 120 can compute other electrical properties (features) of the tissue sample 210, in addition to the Cole relaxation frequency, that are based on the electrical properties of the tissue sample 210. In some embodiments, the classification module 120 can generate an impedance spectrum or multiple impedance spectra for a set of computed electrical impedances. A given impedance spectrum indicates the magnitude of electrical impedances at various locations of the tissue sample 210 as a function of the operating frequency of the current injected during measurement. In some embodiments, the classification module 120 transmits electrical measurement data (e.g., the measured voltages and currents) and/or computed data (e.g., the electrical impedances, impedance spectrums, features, diagnosis predictions, etc.) to the display 140 and/or the I/O units 150. For example, the classification module 120 can determine various properties (e.g., the impedance spectra) or determine various probabilities from the impedance spectra computed by the classification module 120. In various embodiments, the classification module 120 can classify the presence or absence of inflammation, scarring, carcinomas, and/or other types of features present in the tissue sample 210.

In one example, the classification module 120 can compute the probability of malignant cancer cells based on one or more features. In such instances, each frequency range of the Cole relaxation frequency can indicate that the cancerous cells 214 are more malignant and can indicate a need for more aggressive treatment. An initial cancer-detection threshold for breast cancer cells can be 100 kHz. A Cole relaxation frequency occurring within a first critical range of 100 kHz to 600 kHz can indicate that the breast cancer may recur after treatment. A Cole relaxation frequency occurring within a second critical range above 600 kHz may indicate a high likelihood of metastasis after treatment. The cancer-detection threshold and the number and thresholds for each of the critical ranges can vary for each type of cancer (e.g., cancer cells in the pancreas, lung, gastrointestinal tract, etc.). In such instances, the classification module 120 can compute the Cole relaxation frequency for a set of impedances by performing a regression analysis to find a best fit to pre-determined impedance spectrums stored in the memory 122. Other features have been used to determine the malignancy of cancer cells, and accuracy to determine the state of cancer cells is more statistically relevant at high frequencies, above 10-20 MHz.

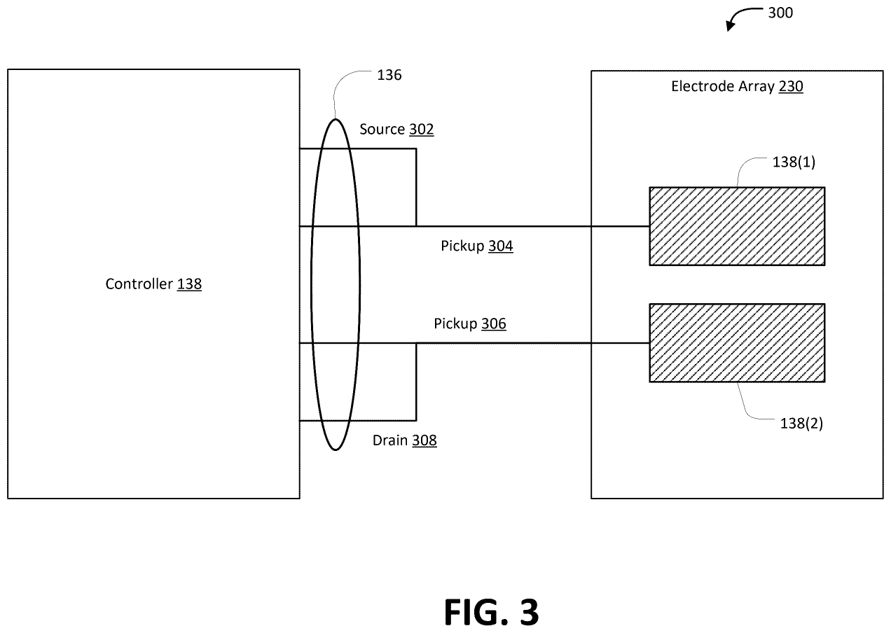

FIG. 3 illustrates a bipolar configuration 300 for a pair of electrodes 138(1)-138(2) that can be included in the tissue measurement system 100 of FIG. 1, according to various embodiments. As shown, the bipolar configuration 300 includes, without limitation, the controller 134, an electrode array 230, a source connector 302, pickup connectors 304, 306, and a drain 308. The electrode array 230 includes, without limitation, the electrode pair including the electrodes 138(1)-138(2).

In operation, the electrode array 230 includes at least one electrode pair formed from the electrodes 138(1)-138(2). In various embodiments, the electrode pair is connected to the controller 134 in a bipolar configuration via a set of connectors 136. The set of connectors includes a source connector 302, pickup connectors 304, 306, and a drain connector 308. The electrode pair in the bipolar configuration is connected to both a voltage measuring circuit and a current measuring circuit included in the measuring circuit 240 of the controller 134. Alternatively, in some embodiments, the electrodes 138(1)-138(2) are included in a tetrapolar configuration, where the source connector 302 is connected to an additional electrode 138 (e.g., 138(3)) and the drain is connected to an additional electrode (e.g., 138(4)) as well. In operation, the electrodes 138(1)-138(2) operating in the bipolar configuration exhibit the same behavior as an electrode column 138(1)-138(4) in a tetrapolar configuration when the tissue sample 210 contacts all the applicable electrodes, including the electrodes 138(3)-138(4) when using the tetrapolar configuration.

Advantageously, the bipolar configuration enables the miniaturization of the electrodes 138(1)-138(2), which would not be possible for electrodes 138(1)-138(4) in the tetrapolar configuration or other configurations that include additional electrodes 138. For example, one requirement for electrodes 138(1)-138(4) in a tetrapolar configuration is that the two pickup electrodes (e.g., electrodes 138(1)-138(2)) be placed between the source electrode 138(3) and the drain electrode 138(4). Such a requirement limits the minimum distance of the electrodes 138(1)-138(4) that are in use. As the tissue sample 210 must contact the source electrode 138(3) and the drain electrode 138(4) in the tetrapolar configuration, the size of the tissue sample 210 must be large enough to cover the distance between the source electrode 138(3) and the drain electrode 138(4). In the bipolar configuration, the tissue sample 210 can be as small as the smallest distance between two adjacent electrodes 138(1)-138(2).

Further, the tissue measurement tool 130 connecting the controller 134 to the electrodes 138(1)-138(2) reduces the number of wires connecting the controller 134 to the electrodes 138(1)-138(2). For example, the source connector 302 and the pickup connector 304 can be connected within the controller 134 or in a portion of a wire. In such instances, a single wire can connect the controller 134 to the electrode 138(1). Similarly, the drain connector 308 and the pickup connector 306 can be connected within the controller 134 or in a portion of a wire. In such instances, a single wire can connect the controller 134 to the electrode 138(2). In addition, the controller 134 can include simple electronics to connect to the electrodes 138(1)-138(2). For example, the electronics used to measure the voltage at the pickup 306 and at the drain 308 are redundant and can be reduced to a single set of voltage-sensing electronics.

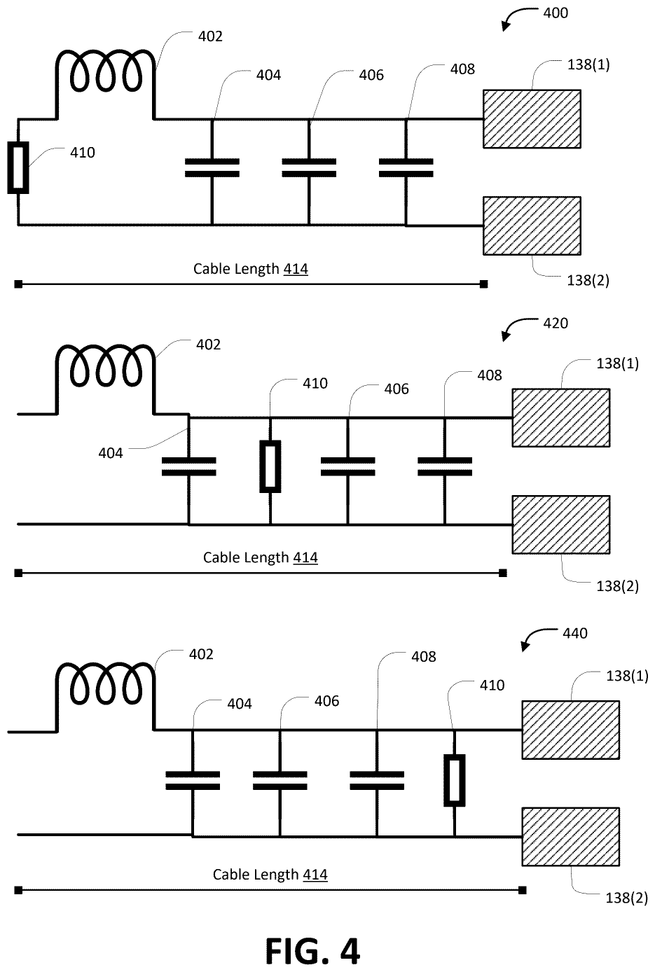

FIG. 4 illustrates exemplar configurations for a decoupling device 410 that can be coupled to the electrodes 138 of the tissue measurement system 100 of FIG. 1, according to various embodiments. In various embodiments, the electrodes 138(1)-138(2) can be connected to the controller 134 via a set of connectors 136 of a given cable length 414. The set of connectors 136 includes parasitic components, such as a parasitic inductance represented by parasitic inductor 402 and a parasitic capacitance represented by parasitic capacitors 404-408.

Configuration 400 illustrates a set of connectors 136 connecting a pair of electrodes 138(1)-138(2) to a controller. As shown, the set of connectors 136 includes a parasitic inductor 402, one or more parasitic capacitors 404-408, and a decoupling device 410 placed proximate to the controller 134. Configuration 420 illustrates the set of connectors 136 connecting the pair of electrodes 138(1)-138(2) to the controller 134. As shown, the set of connectors 136 includes a parasitic inductor 402, one or more parasitic capacitors 404-408, and the decoupling device 410 placed at a location between the controller 134 and the electrodes 138 within the cable (e.g., within the cable length 414). Configuration 440 illustrates a set of connectors 136 connecting the pair of electrodes 138(1)-138(2) to a controller. As shown, the set of connectors 136 includes a parasitic inductor 402, one or more parasitic capacitors 404-408, and a decoupling device 410 placed proximate to the electrodes 138(1)-138(2).

In various embodiments, the tissue measurement tool 130 includes a decoupling device 410 that is connected to the electrodes 138(1)-138(2) in parallel. In such instances, the decoupling device 410 is configured to enable the classification module 120 to record the parasitic inductance and/or the parasitic capacitance as the tissue measurement tool impedance Z_{d}. In some embodiments, the decoupling device 410 is a resistor that connects to the pickups 304, 306. For example, the decoupling device 410 can have a specific resistance of 10 and 5000 Ohms. In some embodiments, the decoupling device 410 is connected to the electrodes 138(1)-138(2) when the electrodes 138(1)-138(2) are connected in the bipolar configuration, the tetrapolar configuration, and/or other configurations.

At least one advantage of a tissue measurement tool 130 that includes the decoupling device 410 is that the decoupling device 410 limits the maximum impedance of the system to the impedance of the decoupling device 410. In various embodiments, the tissue measurement tool 130 exhibits a linear detection range below a threshold value. Without the decoupling device 410, the determined impedance can deviate from ideal behavior in a non-linear fashion if the tissue impedance is above a threshold value of the tissue measuring tool 130. By contrast, the inclusion of the decoupling device 410 causes the total impedance Zₘ to have a maximum value equal to the impedance of the decoupling device 410, keeping the determined impedance within the linear detection range of the tissue measurement tool 130, even if the tissue sample 210 has an electrical impedance that is far higher (e.g., an electrical impedance ten times higher than the impedance of the decoupling device 410).

Alternatively, in some embodiments, the decoupling device 410 can be a variable resistor. In such instances, the decoupling device 410 can be modified to a resistance set by the controller. For example, the tissue measurement tool 130 includes a linear detection range, such as a linear detection range between the operating frequencies of 1 kHz and 50 MHz, where the tissue impedance tool effectively detects electrical impedances. In such instances, the tissue measurement system 100 can select a decoupling device 410 with an impedance such that, when combined with the impedance of the tissue sample 210, the resulting impedance at a given frequency is a constant value for which the tissue measurement tool 130 has an ideal response. Accordingly, the decoupling device 410 having a variable resistance allows the tissue measurement system 100 to adjust the impedance and then compare impedances from different tissues, or locations of the same tissue, more accurately, as any residual parasitic components are essentially identical, regardless of the impedance of the tissue.

In various embodiments, the impedance value of the decoupling device 410 can be changed for each measurement of the tissue sample 210 by first measuring the tissue sample 210 using a standard decoupling value to determine the total impedance. In such instances, the tissue measurement system 100 can calculate the most suitable impedance value for the decoupling device 410 (“decoupling value”) via the processor 132. The tissue measurement tool 130 can then measure the tissue sample 210 again using the optimal decoupling value, thus obtaining an optimized tissue impedance. At least one technical advantage of optimizing the impedance value of the decoupling device 410 is that measurements of the electrical properties of the tissue sample 210 are significantly more comparable to other tissue samples 210, even when the computed tissue impedances greatly differ. Moreover, the determination of an ideal impedance value of the decoupling device 410 can ensure that the measurements are obtained within the linear detection range of the tissue measurement tool 130.

In various embodiments, the decoupling device 410 is placed at various locations along the cable length 414. For example, in the configuration 400, the decoupling device 410 is located near the controller 134 (e.g., before the parasitic inductor 402). By contrast, in the configuration 420, the decoupling device 410 is located near the middle of the connectors 136 (e.g., between the parasitic inductors 404 and 406). In the configuration 440, the decoupling device 410 is located near the electrodes 138-138(2) (e.g., after the parasitic capacitor 408). In various embodiments, the location of the decoupling device 410 along the cable does not affect how the decoupling device 410 removes the parasitic impedances. For example, the location of the decoupling device 410 can match that of configuration 400, where the decoupling device 410 is proximate to the controller 134.

The addition of the decoupling device 410 limits the effect of the parasitic elements 402-408, enables the recording of such parasitic elements for further removal from the resulting impedance, and enables the electrode array 230 to connect to the controller 134 using longer cables (e.g., a cable length 414 above 2 meters), such as extendable tethers. In particular, for wires that include a decoupling device 410 having variable impedance values, the resulting impedances associated with longer cable lengths 414 can be more reliable. In such instances, the tissue measurement system 100 can include the electrodes 138(1)-138(2) on longer probes, expanding the types of tissue measurements to include both ex vivo and in vivo measurement of tissue samples 210. For example, the electrodes 138(1)-138(2) can be at the ends of a long probe that can be extended into a patient and the tissue sample 210 can still be attached to the patient. Moreover, one advantage of the decoupling device 410 is that the decoupling device 410 can be placed anywhere along the cable 414 without limitations to its application. For example, the decoupling device 410 is effective when the electrodes 138(1) and 138(2) are placed at distal location of a catheter used for in-vivo measurements. In some embodiments, the decoupling device 410 can be placed near the controller 134 at the base of cable 414 (e.g., the location of the decoupling device 410 in the configuration 400).

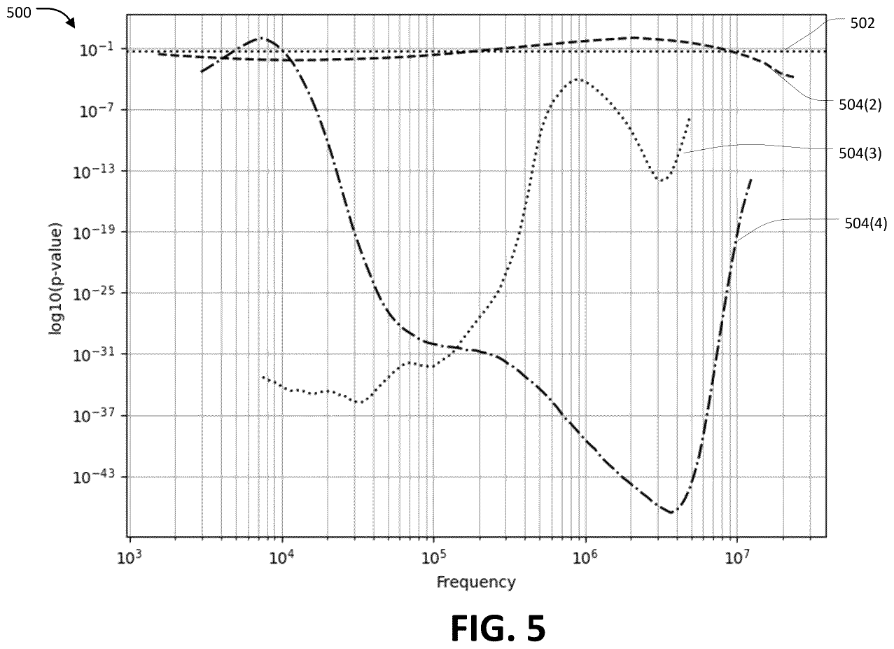

FIG. 5 is a graph 500 illustrating different features extracted from the tissue impedances measured by a tissue measurement tool 130 that does not include a decoupling device 410, according to various embodiments. As shown, the graph 500 includes a threshold 502 and a set of Mann-Whitney p-values 504 (e.g., 504(1)-504(2)).

As shown, the graph 500 illustrates a result of a Mann-Whitney analysis for each point of the frequency of impedance spectra obtained by the tissue measurement tool 130 in the absence of the decoupling device 410. The tissue measurement system 100 determines impedances for tissue samples that include carcinoma (positive) or healthy tissue (negative). The threshold line 502 represents the threshold value (0.05) below which p-values are statistically relevant. The lines 504(1), 504(2), 504(3) represent Mann-Whitney p-values of three features extracted from the impedance spectra that the tissue measurement system 100 processed. As the tissue measurement system 100 does not include a decoupling device 410, the tissue measurement system 100 processes the impedance spectra without removing the parasitic capacitance. The graph 500 thus illustrates that the statistical relevance of discriminating between positive and negative carcinoma sharply decreases above 3 MHz (i.e., 3*10⁶ Hz). In the case of feature represented by the line 504(1), the p-values are greater than the statistically relevant threshold of 0.05 depicted by line 502. The lines 504(3)-504(4) also demonstrate clearly that in the absence of the decoupling device 410, the parasitic capacitance of the system 100 dramatically diminishes any discrimination between carcinoma (positive) and healthy tissue (negative) at high frequencies, namely above 2 MHz.

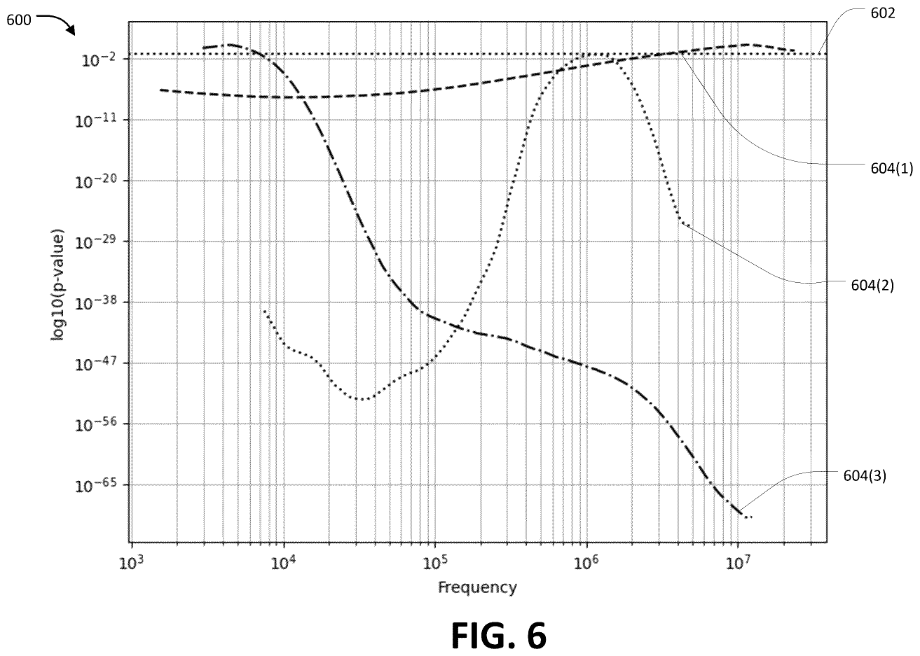

FIG. 6 is a graph illustrating different features extracted from the tissue impedances measured by a tissue measurement tool 130 that includes a decoupling device 410, according to various embodiments. As shown, the graph 600 includes a threshold 602 and a set of Mann-Whitney p-values 604 (e.g., 604(1)-604(3)).

As shown, the graph 600 illustrates a result of a Mann-Whitney analysis for each point of the frequency of impedance spectra obtained by the tissue measurement tool 130 that includes the decoupling device 410. The threshold 602 represents a value (0.05) below which the p-values are statistically relevant. The Mann-Whitney p-values 604 are for three particular features extracted from the impedance spectra that have been processed. In various embodiments, the classification module 120 can extract the features by removing the parasitic capacitance included in the tissue measurement system 100 using the decoupling device 410.

Graph 600 shows that at higher frequencies, the statistical difference between positive and negative carcinoma increases exponentially. Graph 600 demonstrates clearly that the decoupling device 410 effectively removes the parasitic capacitance of the tissue measurement system 100 overall. Consequently, the tissue measurement system 100 can compute the impedances of tissue samples at high frequencies (e.g., as high as 100 MHz) with high accuracy. Moreover, the lines 604(1)-604(3) in the graph 600 demonstrate that cancer detection is statistically more accurate when the impedances of tissue samples are acquired at frequencies above 10 MHz. In contrast with the graph 500, this statistical difference between positive and negative carcinoma would not be possible when the tissue measurement tool 130 acquires measurements when the decoupling device 410 is absent. The effective removal of the parasitic contribution of the tissue measurement tool 130 via the decoupling device 410 demonstrates that the impedance measurements at frequencies above 10 MHz can discriminate between carcinoma and healthy tissue. Moreover, the removal of the parasitic contribution of the tissue measurement tool 130 enables the tissue measurement tool 130 to extract various features at high frequencies.

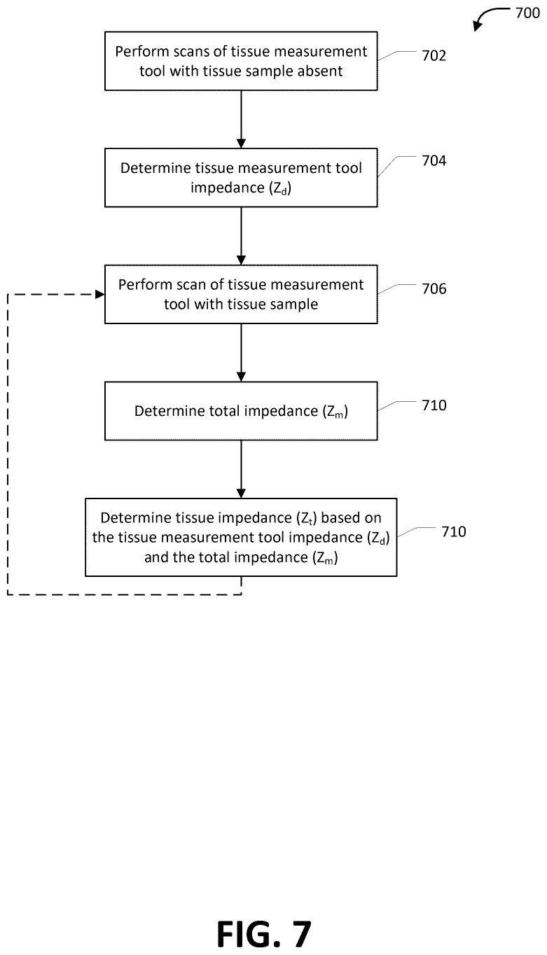

FIG. 7 sets forth a flowchart of method steps for measuring tissue impedances using a bipolar electrode configuration, according to various embodiments. Although the method steps described in conjunction with the systems of FIGS. 1-6, persons skilled in the art will understand that any system configured to perform the method steps, in any order, is within the scope of the present disclosure.

As shown, the method 700 begins at step 702, where the tissue measurement system 100 performs a scan of the tissue measurement tool 130 where the tissue sample 210 is absent. In various embodiments, the tissue measurement tool 130 includes a decoupling device 410 that has an impedance such that the total impedance of the tissue measurement tool 130, when connected to the tissue sample 210, falls within the linear detection range of the tissue measurement tool 130. The electrodes 138 of the tissue measurement tool 130 and the decoupling device 410 are connected in parallel via the set of connectors 136. The classification module 120 generates commands for the tissue measurement tool 130 to measure voltages and currents at one or more operating frequencies when the electrodes 138 in an electrode array 230 in the absence of a tissue sample 210. In such instances, the scan of the tissue measurement tool 130 absent the tissue sample 210 enables the classification module 120 to record the parasitic contributions for the tissue measurement tool 130.

At step 704, the tissue measurement system 100 determines the tissue measurement tool impedance Z_{d}. In various embodiments, the classification module 120 receives the measured voltages and currents from the tissue measurement tool 130 and computes the impedance of the tissue measurement tool 130 in the absence of the tissue sample 210. The tissue measurement tool impedance Z_{d} as computed contains the total parasitic contributions of the tissue measurement tool 130. For example, the parasitic contributions can include the parasitic capacitance (e.g., the parasitic capacitors 404-408) and the parasitic inductance (e.g., the parasitic inductor 402).

At step 706, the tissue measurement system 100 performs a scan of the tissue measurement tool 130 while connected to a tissue sample 210. In various embodiments, the classification module 120 generates and transmits commands to the tissue measurement tool 130 to measure voltages and currents through one or more sections of the tissue sample 210. For example, the controller 134 may perform a series of electrical measurements on a section of the tissue sample 210 for 10 to 60 seconds. The controller 134 may then cause the measuring circuit 240 to connect to a different electrode pair to perform an additional series of electrical measurements on a different section of the tissue sample 210 for 10 to 60 seconds. In some embodiments, the controller 134 can measure electrical properties for all sections of the tissue sample 210 within the electrode array 230 in under 60 to 120 seconds.

At step 708, the tissue measurement system 100 determines the total impedance Zₘ. In various embodiments, the classification module 120 receives the measured voltages and currents from the tissue measurement tool 130 and computes the impedance of a circuit formed by the tissue measurement tool 130 and a connected tissue sample 210. The total impedance Zₘ includes both the tissue measurement tool impedance Z_{d} and the tissue impedance Zₜ. In such instances, the tissue measurement tool impedance Z_{d} is in parallel with the tissue impedance Zₜ.

At step 710, the tissue measurement system 100 determines the tissue impedance Zₜ. In various embodiments, the classification module 120 computes the tissue impedance Zₜ based on one or more of the tissue measurement tool impedance Z_{d} and the total impedance Zₘ. For example, the classification module 120 can compute the tissue impedance Zₜ based on the parallel circuit configuration of the tissue measurement tool impedance Z_{d} and the tissue impedance Zₜ. In various embodiments, the tissue measurement system 100 can store the computed tissue impedance Zₜ. In some embodiments, the classification module 120 uses the computed tissue impedance Zₜ to determine one or more other properties of the tissue sample 210. For example, the classification module 120 can compare the tissue impedance Zₜ to one or more thresholds to determine whether the section of the tissue sample contains cancerous cells 214. In another example, the classification module 120 can compute a series of tissue impedances Zₜ to generate impedance spectra for the tissue sample 210. Upon determining the tissue impedance Zₜ, the tissue measurement system 100 can optionally return to step 706 to measure another tissue sample 210.

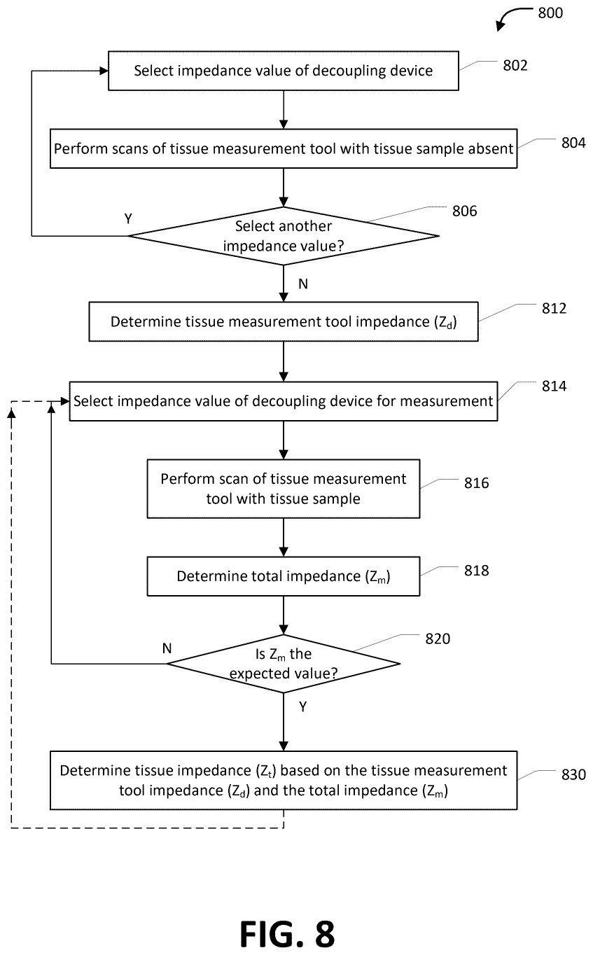

FIG. 8 sets forth a flowchart of method steps for measuring tissue impedances using a bipolar electrode configuration and a decoupling device having a variable impedance, according to various embodiments. Although the method steps described in conjunction with the systems of FIGS. 1-6, persons skilled in the art will understand that any system configured to perform the method steps, in any order, is within the scope of the present disclosure.

Method 800 begins at step 802, where the tissue measurement system 100 selects an impedance value for a decoupling device 410 included in the tissue measurement tool 130. In various embodiments, the tissue measurement tool 130 includes a linear detection range, such as a linear detection range between 1 kHz and 50 MHz. In such instances, the controller 134 of the tissue measurement system 100 selects an impedance value for the decoupling device 410, where the impedance value is within a linear detection range. For example, an impedance value can be selected that is within a range of 1 to 10,000 Ohms. In some embodiments, the impedance value is based on an ideal response of the tissue measurement tool 130.

At step 804, the tissue measurement system 100 performs a scan of the tissue measurement tool 130 where the tissue sample 210 is absent. In various embodiments, the tissue measurement system iteratively scans the tissue measurement tool 130 at all possible impedance values for the decoupling device 410. Each scan is performed where the tissue sample 210 is absent, enabling the tissue measurement system 100 to determine parasitic contributions for each possible impedance value. The electrodes 138 of the tissue measurement tool 130 and the decoupling device 410 are connected in parallel via the set of connectors 136. The classification module 120 generates commands for the tissue measurement tool 130 to measure voltages and currents at one or more operating frequencies when the electrodes 138 in an electrode array in the absence of a tissue sample 210.

At step 806, the tissue measurement system 100 determines whether to select another impedance value for the decoupling device 410. In various embodiments, the tissue measurement system 100 determines whether a scan has been performed for each possible impedance value of the decoupling device 410. When the classification module 120 of the tissue measurement system 100 determines that a scan of the tissue measurement tool 130 for at least one impedance value of the decoupling device 410 needs to be performed, the classification module 120 causes the tissue measurement system 100 to return to step 802 to select another impedance value for the decoupling device 410. Otherwise, the classification module 120 determines that the sweep of scans of the tissue measurement tool 130 for all possible impedance values of the decoupling device 410 has been completed and causes the tissue measurement system 100 to proceed to step 812.

At step 812, the tissue measurement system 100 determines the tissue measurement tool impedance Z_{d}. In various embodiments, the classification module 120 receives the measured voltages and currents from the tissue measurement tool 130 and computes the impedance of the tissue measurement tool 130 in the absence of the tissue sample 210. The tissue measurement tool impedance Z_{d} as computed contains the total parasitic contributions of the tissue measurement tool 130. For example, the parasitic contributions can include the parasitic capacitance (e.g., the parasitic capacitors 404-408) and the parasitic inductance (e.g., the parasitic inductor 402).

At step 814, the tissue measurement system 100 selects an impedance value of the decoupling device 410 for use in measurement. In various embodiments, the classification module 120 can cause the controller 134 to select a specific impedance value for the decoupling device 410 when measuring a tissue sample 210. For example, the tissue measurement tool 130 includes a linear detection range, such as a linear detection range between the operating frequencies of 1 kHz and 50 MHz, where the tissue impedance tool effectively detects electrical impedances. In such instances, the tissue measurement system 100 can select a decoupling device 410 with an impedance such that, when combined with the impedance of the tissue sample 210, the resulting impedance at a given frequency is a constant value for which the tissue measurement tool 130 has an ideal response.

At step 816, the tissue measurement system 100 performs a scan of the tissue measurement tool 130 while connected to a tissue sample 210. In various embodiments, the classification module 120 generates and transmits commands to the tissue measurement tool 130 to measure voltages and currents through one or more sections of the tissue sample 210. For example, the controller 134 may perform a series of electrical measurements on a section of the tissue sample 210 for 10 to 60 seconds. The controller 134 may then cause the measuring circuit 240 to connect to a different electrode pair to perform an additional series of electrical measurements on a different section of the tissue sample 210 for 10 to 60 seconds. In some embodiments, the controller 134 can measure electrical properties for all sections of the tissue sample 210 within the electrode array 230 in under 60 to 120 seconds.

At step 818, the tissue measurement system 100 determines the total impedance Zₘ. In various embodiments, the classification module 120 receives the measured voltages and currents from the tissue measurement tool 130 and computes the impedance of a circuit formed by the tissue measurement tool 130 and a connected tissue sample 210. The total impedance Zₘ includes both the tissue measurement tool impedance Z_{d} and the tissue impedance Zₜ. In such instances, the tissue measurement tool impedance Z_{d} is in parallel with the tissue impedance Zₜ.

At step 820, the tissue measurement system 100 determines whether the total impedance Zₘ is at an expected value. In various embodiments, the classification module 120 can analyze the total impedance Zₘ to ascertain whether the total impedance Zₘ is within an ideal linear range of the tissue measurement tool 130. The ideal value of tissue measurement tool impedance Z_{d} is such that the total impedance Zₘ at a given frequency, or multiple discriminating frequencies, falls within the linear range of the tissue measurement tool 130. If the classification module 120 determines that the value or values of Zₘ are not an expected value, the tissue measurement system 100 returns to step 814 to select another impedance value for the decoupling device 410, thereby changing the value of the tissue measurement tool impedance Z_{d}. Otherwise, the classification module 120 determines that the value or values of Zₘ are an expected value and proceeds to step 830.

At step 830, the tissue measurement system 100 determines the tissue impedance Zₜ. In various embodiments, the classification module 120 computes the tissue impedance Zₜ based on one or more of the tissue measurement tool impedance Z_{d} and the total impedance Zₘ. For example, the classification module 120 can compute the tissue impedance Zₜ based on the parallel circuit configuration of the tissue measurement tool impedance Z_{d} and the tissue impedance Zₜ. In various embodiments, the tissue measurement system 100 can store the computed tissue impedance Zₜ. In some embodiments, the classification module 120 uses the computed tissue impedance Zₜ to determine one or more other properties of the tissue sample 210. For example, the classification module 120 can compare the tissue impedance Zₜ to one or more thresholds to determine whether the section of the tissue sample contains cancerous cells 214. In another example, the classification module 120 can compute a series of tissue impedances Zₜ to generate impedance spectra for the tissue sample 210. Upon determining the tissue impedance Zt, the tissue measurement system 100 can optionally return to step 814 to measure another tissue sample 210.

In sum, the tissue measurement system disclosed herein enables electrical characterization cells to be detected automatically within a sample of tissue based on measured impedances of sections of the tissue. The tissue measurement system includes a tissue measurement tool and a classification module. The tissue measurement tool includes a controller, a decoupling device, and an electrode array. The decoupling device removes parasitic impedances associated with components of the tissue measurement tool and the electrodes. In some embodiments, the decoupling device has a single impedance value. Alternatively, in some embodiments, the decoupling device has a variable impedance value that is selected based on the tissue sample that is to be measured. The classification module receives measured voltages and currents the tissue measurement tool in the absence of the tissue sample and computes a tissue measurement tool impedance. The classification module computes a tissue impedance based on both the tissue measurement tool impedance and the total impedance. In various embodiments, the classification module compares the tissue impedance to one or more predetermined thresholds, where a tissue impedance that exceeds at least one of the one or more predetermined thresholds indicates that cancerous cells are present in the section of the tissue sample.

In some embodiments, the electrode array includes an electrode pair connected in a bipolar configuration. The first electrode in the electrode pair is a stimulating electrode that transmits current through a section of a tissue sample, and a second electrode in the electrode pair is a drain that receives the current through the tissue sample. The electrodes are connected in a current measurement circuit and a voltage measurement circuit. The tissue measurement tool measures voltages and currents section of the tissue sample that is connected to the electrodes based on the current injected at an operating frequency. The classification module receives the measured voltages and currents for one or more frequencies and computes a total impedance for circuit formed by the section of the tissue sample and the tissue management tool.

At least one technical advantage of the disclosed design relative to the prior art is that the disclosed design enables a tissue measurement system to be employed for bioelectrical impedance spectroscopy over a wider range of frequencies relative to the effective frequency ranges of conventional tissue measurement systems. In particular, the disclosed design includes a decoupling device that enables the removal of parasitic impedances, which are otherwise generated by the components of the device and electrodes, from the impedances measured by the tissue measurement tool. A tissue measurement system that incorporates the disclosed design can achieve greater impedance accuracy over a wider range of frequencies, such as frequencies above 2 MHz to 100 MHz. Consequently, a tissue measurement system that incorporates the disclosed design can thus distinguish between additional types of cancerous cells and noncancerous cells. Further, various embodiments of the disclosed design use a bipolar configuration of electrode pairs in an electrode array. A tissue measurement system that incorporates the disclosed design thus requires fewer electrodes when measuring the voltages and currents associated with given tissue samples. These technical advantages provide one or more technological improvements over prior art approaches.
1. In various embodiments, a system for determining electrical properties of tissue samples comprises a tissue measurement tool comprising at least two electrodes that measure, while operating at a frequency, a set of electrical properties corresponding to a section of tissue, and a decoupling device connected to the at least two electrodes, and a classification module that computes, based on the set of electrical properties, at least an electrical impedance of the section of tissue.
2. The system of clause 1, where the at least two electrodes are connected in a bipolar configuration.
3. The system of clause 1 or 2, where an electrical impedance of the decoupling device falls within a linear detection range of the tissue measurement tool.
4. The system of any of clauses 1-3, where the decoupling device reduces parasitic impedances associated with the tissue measurement tool at frequencies above 20 MHz.
5. The system of any of clauses 1-4, further comprising a wire of at least 1 meter that is coupled to the tissue measurement tool.
6. The system of any of clauses 1-5, further comprising an electrode array that includes the at least two electrodes.
7. The system of any of clauses 1-6, where electrodes included in the electrode array are interdigitated.
8. The system of any of clauses 1-7, where the classification module further extracts, from the set of electrical properties at frequencies above 1 kHz, one or more features, and determines, based the one or more features, that the section of the tissue contains cancerous cells.
9. The system of any of clauses 1-8, where the section of tissue comprises a portion of an excised tissue sample.
10. The system of any of clauses 1-9, where the section of tissue remains attached to a patient, and the set of electrical properties is measured in vivo.
11. In various embodiments, a method for determining electrical properties of tissue samples comprises measuring, by a tissue measurement tool while operating at a frequency, a set of electrical properties corresponding to a section of tissue, wherein the tissue measurement tool includes, at least two electrodes, and a decoupling device connected to the at least two electrodes, and computing, by a classification module based on the set of electrical properties, at least an electrical impedance of the section of tissue.
12. The method of clause 11, further comprising extracting, by the classification module, from the set of electrical properties at frequencies above 1 kHz, one or more features, and determining, by the classification module and based the one or more features, that the section of the tissue contains cancerous cells.
13. The method of clause 11 or 12, where the one or more features includes at least one feature at a frequency above 10 MHz.
14. The method of any of clauses 11-13, further comprising measuring, by the tissue measurement tool while the tissue is absent, an additional set of electrical properties corresponding to the tissue measurement tool, and computing, by the classification module and based on the additional set of electrical properties, an electrical impedance of the tissue measurement tool, wherein the classification module computes the electrical impedance of the section of tissue based on the electrical impedance of the tissue measurement tool.
15. The method of any of clauses 11-14, where the at least two electrodes are connected in a bipolar configuration.
16. The method of any of clauses 11-15, where an electrical impedance of the decoupling device falls within a linear detection range of the tissue measurement tool.
17. The method of any of clauses 11-15, where the tissue measurement tool further includes an electrode array that includes the at least two electrodes.
18. The method of any of clauses 11-17, where electrodes included in the electrode array are interdigitated.
19. The method of any of clauses 11-18, where the decoupling device reduces parasitic impedances associated with the tissue measurement tool at frequencies above 20 MHz.
20. The method of any of clauses 11-19, where the section of tissue remains attached to a patient, and the set of electrical properties is measured in vivo.

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A system for determining electrical properties of tissue samples, the system comprising:
a tissue measurement tool comprising:
at least two electrodes that measure, while operating at a frequency, a set of electrical properties corresponding to a section of tissue, and
a decoupling device connected to the at least two electrodes; and
a classification module that computes, based on the set of electrical properties, at least an electrical impedance of the section of tissue.

2. The system of claim 1, wherein the at least two electrodes are connected in a bipolar configuration.

3. The system of claim 1, wherein an electrical impedance of the decoupling device falls within a linear detection range of the tissue measurement tool.

4. The system of claim 1, wherein the decoupling device reduces parasitic impedances associated with the tissue measurement tool at frequencies above 20 MHz.

5. The system of claim 1, further comprising a wire of at least 1 meter that is coupled to the tissue measurement tool.

6. The system of claim 1, further comprising an electrode array that includes the at least two electrodes.

7. The system of claim 6, wherein electrodes included in the electrode array are interdigitated.

8. The system of claim 1, wherein the classification module further:
extracts, from the set of electrical properties at frequencies above 1 kHz, one or more features; and
determines, based the one or more features, that the section of the tissue contains cancerous cells.

9. The system of claim 8, wherein the section of tissue comprises a portion of an excised tissue sample.

10. The system of claim 8, wherein the section of tissue remains attached to a patient, and the set of electrical properties is measured in vivo.

11. A method for determining electrical properties of tissue samples, the method comprising:
measuring, by a tissue measurement tool while operating at a frequency, a set of electrical properties corresponding to a section of tissue, wherein the tissue measurement tool includes:
at least two electrodes, and
a decoupling device connected to the at least two electrodes; and
computing, by a classification module based on the set of electrical properties, at least an electrical impedance of the section of tissue.

12. The method of claim 11, further comprising:
extracting, by the classification module, from the set of electrical properties at frequencies above 1 kHz, one or more features; and
determining, by the classification module and based the one or more features, that the section of the tissue contains cancerous cells.

13. The method of claim 12, wherein the one or more features includes at least one feature at a frequency above 10 MHz.

14. The method of claim 11, further comprising:
measuring, by the tissue measurement tool while the tissue is absent, an additional set of electrical properties corresponding to the tissue measurement tool; and
computing, by the classification module and based on the additional set of electrical properties, an electrical impedance of the tissue measurement tool, wherein the classification module computes the electrical impedance of the section of tissue based on the electrical impedance of the tissue measurement tool.

15. The method of claim 11, wherein the at least two electrodes are connected in a bipolar configuration.

16. The method of claim 15, wherein an electrical impedance of the decoupling device falls within a linear detection range of the tissue measurement tool.

17. The method of claim 11, wherein the tissue measurement tool further includes an electrode array that includes the at least two electrodes.

18. The method of claim 17, wherein electrodes included in the electrode array are interdigitated.

19. The method of claim 11, wherein the decoupling device reduces parasitic impedances associated with the tissue measurement tool at frequencies above 20 MHz.

20. The method of claim 11, wherein the section of tissue remains attached to a patient, and the set of electrical properties is measured in vivo.
